SEQUENCE LISTING (4 sequences)

SEQ ID NO: 1 - protein, 50 aa, Homo sapiens
VMLKKKQYTSIHHGVVEVDAAVTPEERHLSKMQQNGYENPTYKFFEQMQN

SEQ ID NO: 2 - protein, 99 aa, Homo sapiens
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - protein, 6 aa, Homo sapiens
ITLVML

SEQ ID NO: 4 - protein, 12 aa, Homo sapiens
VIVITLVMLKKK

(19) United States
(12) Patent Application Publication
(10) Pub. No.: US 2003/0022251 A1
(43) Pub. Date: Jan. 30, 2003

(54) Gamma-secretase in vitro screening assay

(75) Inventors: Klaus Fuchs, Schemmerhofen (DE); Cornelia Dorner-Ciossek, Ravensburg (DE); Marcus Kostka, Mainz (DE); Christian Haass, Icking (DE); Harald Steiner, Muenchen (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/183,119

(22) Filed: Jun. 26, 2002

Related U.S. Application Data
(60) Provisional application No. 60/306,123, filed on Jul. 17, 2001.

(30) Foreign Application Priority Data
Jul. 4, 2001 (DE) DE 101 31 899.5

(51) Int. Cl. G01N 33/567; C12Q 1/37

(52) U.S. Cl. 435/7.21; 435/23

(57) ABSTRACT

According to the screening method of the invention substances capable of modulating γ-secretase activity are identified by incubating a source and a substrate of γ-secretase activity with a test substance and detecting a C-terminal fragment released from said substrate.

### RELATED APPLICATIONS

Benefit of U.S. Provisional Application Serial No. 60/306,123, filed on Jul. 17, 2001 is hereby claimed. Said Provisional Application is herein incorporated by reference in its entirety.

### FIELD OF THE INVENTION

The invention relates to a novel peptide playing an important role in Alzheimer's disease, to screening assays and test kits based on the detection of said novel peptide and to the use thereof for the identification of modulators of γ-secretase activity. The invention further relates to inhibitors identified by the above screening assays and to pharmaceutical compositions comprising the inhibitors.

### BACKGROUND OF THE INVENTION

Several documents are cited throughout the text of this specification. Each of the documents cited herein (including any manufacturer's specifications, instructions, etc.) are hereby incorporated by reference; however, there is no admission that any document cited is indeed prior art to the present invention.

Alzheimer's disease (AD) is the most abundant neurodegenerative disorder worldwide. Senile plaques, composed of amyloid β-peptide (Aβ) appear to be a major pathological alteration in the brain of AD patients (Selkoe, 1999). Almost all familial AD (FAD) associated mutations affect the generation of Aβ by increasing the production of the highly amyloidogenic 42 amino acid variant (Selkoe, 1999). Aβ is produced from the β-amyloid precursor protein (βAPP) by endoproteolysis, whereby at least two proteolytic activities are required for Aβ generation: beta-secretase (β-secretase; BACE) mediates the N-terminal cleavage producing a membrane associated C-terminal fragment of βAPP, called C99 or CTFβ (SEQ ID NO:2) (Vassar and Citron, 2000). C99 is the immediate precursor for the next proteolytic processing step: the (presumably intramembraneous) cleavage of C99 by γ-secretase to Aβ and a C-terminal fragment. This cleavage is facilitated by the presenilins, PS1 and PS2, and there is evidence that presenilins themselves could be unusual aspartyl proteases, which mediate the γ-secretase cleavage (Steiner and Haass, 2000; Wolfe and Haass, 2001). An alternative processing pathway of βAPP is initialized by the endoproteolytic activity of alpha-secretase, resulting in a membrane associated C-terminal fragment of βAPP, the fragment having a length of 83 amino acids (C83). Cleavage of C83 by γ-secretase results in the formation of (non-plaque-forming) p3 and a C-terminal fragment.

γ-Secretase cleavage of C99 results in the secretion of Aβ into biological fluids. The C-terminal product of this cleavage, previously described as p7(Haass and Selkoe, 1993) and also called C-terminal fragment gamma (CTFγ), has so far not been observed in vivo. However, it is known that the C-terminus of Aβ is heterogenous in that Aβ may end at position 40 or at position 42 of its precursor C99. Accordingly, two species of CTFγ resulting from γ-secretase cleavage have been expected in the prior art, namely C-terminal fragments consisting of 59 amino acids (CTFγ/59; derived from Aβ40 generation) and consisting of 57 amino acids (CTFγ/57; derived from Aβ42 generation), respectively. Recently, a peptide supposed to be CTFγ/59 or CTFγ/57 has been observed in vitro (McLendon et al., 2000; Pinnix et al., 2001).

From a medical point of view, there is a strong need for means and methods useful for therapeutic intervention in Alzheimer's disease. In this respect, γ-secretase activity, playing a key role in Aβ plaque formation, might be a major target for new therapeutical, especially pharmacological, approaches.

Wolfe et al. (1999) describe an in vitro test system for the assessment of γ-secretase activity. Membranes of cells stably expressing PS1 were prepared. C99 was provided by transfection of the cells with a plasmid encoding C99 fused to the βAPP signal sequence. After incubation, activity of γ-secretase was determined by assessing the amount of Aβ released.

Pinnix et al. (2001) describe experiments designed to detect a C-terminal fragment of βAPP derived from guinea pig brain. The fragment obtained therein is characterized as a peptide consisting of 57 amino acids.

The technical problem underlying the present invention is to provide novel methods and means for the development of therapies of Alzheimer's disease and other neurodegenerative disorders in which Aβ-plaque formation and/or γ-secretase activity is involved.

### SUMMARY OF THE INVENTION

The above problem of the invention is solved by a method for identifying substances capable of modulating the activity of γ-secretase, comprising the steps: providing a source of γ-secretase activity, providing a substrate of γ-secretase activity having a cleavage site corresponding to the naturally occurring cleavage site between amino acid 49 and amino acid 50 of the substrate C99, incubating said source and said substrate with a test substance under conditions allowing enzymatic activity of said γ-secretase activity, and determining γ-secretase activity by detecting the C-terminal fragment (and especially the amount thereof) released due to the proteolytic activity of said γ-secretase activity. The N-terminus of the previously unknown C-terminal fragment corresponds to amino acid 50 of C99.

As will be explained in detail below (Example 1), the invention is based on the surprising finding that an enzymatic cleavage of C99 between its amino acids 49 and 50 is involved in the processing of C99 to Aβ. The inventors have discovered and characterized the product of this processing step, now called CTFγ/50 (amino acid sequence: VMLKKKQYTSIHHGWEVDMVTPEERHLSKMQQNGYENPTYKFFEQMQN; SEQ ID NO:1; N-terminus corresponding to amino acid 50 of C99). They have demonstrated that a γ-secretase activity is responsible for this processing step. Furthermore, it could be shown that CTFγ/50 is an immediate product of γ-secretase activity acting on C99 and not the product of a further degradation of the previously described CTFγ/57 or CTFγ/59. Thus, these results clearly demonstrate that γ-secretase activity acts at a previously unknown cleavage site of C99. The identification of this proteolytic activity offers a novel approach for the pharmaceutical modulation of the enzymatic reactions involved in Aβ production and, thus, for therapeutical intervention.

In the prior art, γ-secretase activity has been determined by measuring release of Aβ (Wolfe et al., 1999; WO 01/16355). However, formation of the known Aβ variants Aβ40 (C-terminus at position 40 of C99) and Aβ42 (C-terminus at position 42 of C99) reflects a cleavage between amino acids 40 and 41 and between amino acids 42 and 43 of C99, respectively. Thus, these methods do not allow the assessment of the proteolytic activity at the cleavage site between amino acids 49 and 50. This difference is of great importance as release of Aβ40 and Aβ42 possibly requires an additional (exo)peptidase activity which might interfere with the γ-secretase activity to be determined. In other words, screening assays for the detection of modulators of γ-secretase activity that are based on the determination of cleavage products which are not the immediate products of said activity but result from additional cleavage events might give misleading data as it is unclear whether said modulators act on said γ-secretase activity or on other enzymatic activities involved in Aβ formation.

CTFγ/50, being a processing product not only in the BACE /γ-secretase pathway but also in the α-secretase/γ-secretase pathway, can be easily assessed in in vitro assays, e.g. by immunoblotting or ELISA. Thus, determination of the amount of CTFγ/50 released can be used as a simple and cost-effective read-out in methods for the identification of substances that modulate γ-secretase activity.

In addition, the regulation of Aβ40 production vis-a-vis Aβ42 production is an unclarified issue. Especially, some candidate inhibitors seem to inhibit Aβ40 release but stimulate Aβ42 secretion. It may be hypothesized that such substances might specifically inhibit an (exo)peptidase acting downstream of γ-secretase activity, i.e. further degrading Aβ42 to Aβ40. The method according to the invention would not be affected by this phenomenon as only substances specifically inhibiting γ-secretase activity would be detected.

Thus, in summary, the method according to the invention allows the specific identification of substances which interfere with the previously unknown cleavage between amino acids 49 and 50 of C99 but not with other endo- or exoproteolytic activities that might be involved in the βAPP processing pathway.

### BRIEF DESCRIPTION OF THE FIGURES

FIG. 1: Identification of in vivo produced CTFγ in human cells and mouse brain according to Example 1: (A) Membrane fractions of HEK 293 cells stably transfected with Swedish mutant βAPP₆₉₅ (swAPP) were analyzed by combined immunoprecipitation/immunoblotting with antibody 6687 to the C-terminus of βAPP. Three βAPP CTFs were detected (C99 (=CTFβ), C83 (=CTFα), and the approximately 6 kDa CTFγ). The same βAPP CTFs including the approximately 6 kDa CTFγ were also observed by immunoblotting with antibody 6687 in mouse brain. (B) The γ-secretase inhibitor DAPT inhibits CTFγ production. HEK 293 cells stably transfected with swAPP were treated with the indicated concentrations of DAPT for 4 h. Upper and middle panel: Membrane fractions were prepared and analyzed for βAPP CTFs by combined immunoprecipitation/immunoblotting with antibody 6687. Increasing concentrations of DAPT lead to a build up of CTFβ and CTFα (upper panel) with a concomitant significant block of CTFγ generation (middle panel). Lower panel: Conditioned media were analyzed for secreted Aβ by combined immunoprecipitation/immunoblotting with antibodies 3926/6E10. The same dose dependent inhibition of CTFγproduction by DAPT was observed for Aβ generation. (C) CTFγ production is dependent on biologically active presenilins. Membrane fractions from control cells expressing PS1 wt or cells stably expressing PS1 D385N were analyzed by combined immunoprecipitation/immunoblotting with antibody 6687. Expression of the non-functional PS1 D385N variant significantly reduces CTFγ production.

FIG. 2: In vitro generation of CTFγ according to Example 1: (A) Time dependent in vitro production of CTFγ. Membrane preparations were incubated at 37° C. for the indicated time points. The reaction mixtures were then separated in a soluble fraction (S100; lower panel) and a pellet fraction (P100; upper panel) by ultracentrifugation. These fractions were immunoblotted with antibody 6687. Note the selective accumulation of CTFγ in the S100(lower panel) fraction after 1-2 h incubation time. Very minor amounts of CTFγ were detected in the P100 fraction, which may be due to sticking of the highly hydrophobic CTFγ to membranes or to contamination of the P100 fraction with minor amounts of soluble proteins. (B) Two independent γ-secretase inhibitors (DAPT and “Compound 1”) inhibit the in vitro production of CTFγ. Membrane preparations were incubated with (+) or without (−) 250 nM DAPT (left panel) or 50 μM CM256 (right panel). The S100 fractions of the reaction mixtures were immunoblotted with antibody 6687. Note that both inhibitors significantly reduce CTFγ generation. (C) In vitro generation of CTFγ depends on biologically active presenilins. Left panel: time dependent in vitro production of CTFγ by membrane preparations derived from cells expressing PS1 wt. Right panel: Inhibition of CTFγ production in membrane preparations derived from cells expressing the biologically inactive PS1 D385N mutation. After termination of the in vitro reactions, βAPP CTFs were identified by immunoblotting with antibody 6687. (D) The γ-secretase inhibitor DAPT reduces the remaining in vitro CTFγ production observed in C (right panel). Left panel: time dependent in vitro production of CTFγ by membrane preparations derived from cells expressing PS1 wt in the presence (+) or absence (−) of 250 nM DAPT. Right panel: Inhibition of CTFγ production in membrane preparations derived from cells expressing the biologically inactive PS1 D385N mutation in the presence (+) or absence (−) of 250 nM DAPT. After termination of the in vitro reactions, βAPP CTFs were identified by immunoblotting with antibody 6687.

FIG. 3: Radiosequencing and mass spectrometry analysis of CTFγ as described in Example 1. (A) CTFγ generated in vitro from membrane preparations of ³⁵S-methionine labeled HEK 293 cells stably expressing swAPP was subjected to radiosequencing. A major methionine peak was observed at cycle 2 and a second peak at cycle 32 of the Edman degradation. The corresponding amino acid sequence of CTFγ starting at valine 50 is shown below. (B) In vivo detection of a truncated CTFγ in living HEK 293 cells stably overexpressing swAPP. Cell lysates from HEK 293 cells stably overexpressing swAPP or a recombinant CTFγ starting at amino acid 43 were co-migrated. CTFs were detected by immunoblotting with antibody 6687. Note that the in vivo produced CTFγ migrates faster than the recombinant fragment.

FIG. 4: Illustration of the similarity of endoproteolytic processing of βAPP and Notch as mentioned in Example 1. Human βAPP is presumably cleaved after position 40, 42 and 49 of the β-amyloid domain. Mouse Notch1 is cleaved PS dependent after amino acid 1743 (Schroeter et al., 1998).

### DETAILED DESCRIPTION OF THE INVENTION

Before describing the invention in greater detail, it should be noted that in the specification and the appended claims, the singular forms “a”, “an” and “the” include plural reference unless the context clearly dictates otherwise. Thus, for example, reference to “a cell line” is a reference to one or more cell lines, and the like.

The amino acid abbreviations are according to the standard one or three letter code. The numbering of the amino acids in Aβ, in other fragments of C99 and in C99 itself is based on C99, i.e. the N-terminal amino acid of C99 is amino acid no. 1 and so on. The sequence of C99 is: DAEFRH DSGYEVHHQKLVFFAEDVGSNKGAI IGLMVGGWIATVIVITLVMLKKK QYTSIHHGWEVDAAVTPEERHLSKMQQNGYENPTYKFFEQMQN (SEQ ID NO:2).

Furthermore, in the specification and in the claims, reference will be made to a number of terms which shall be defined to have the following meanings:

If not indicated otherwise, the term “βAPP” is meant to encompass naturally occurring human full length βAPP, any known splice variant thereof (including e.g. splice variants APP₆₉₅, APP₇₅₁ and APP₇₇₀; cf. e.g. NCBI database entry P05067), any naturally occurring variation or mutation thereof (such as the so-called “swedish mutation” βAPP, swAPP (Citron et al., 1992), and corresponding molecules of other species, as long as they may serve as a substrate for γ-secretase activity in an assay as described hereafter, i.e. comprise a cleavage site as discovered by the inventors. Unless the context clearly dictates otherwise, “βAPP” is also meant to encompass the genuine substrates of γ-secretase activity, i.e. C99 and C83 and respective variants and derivatives. The reason for this definition in the context of this invention is the fact that most assay formats suitable for the method according to the invention comprise endogenous α-secretase- and/or β-secretase activity. Thus, C99 and C83 can be provided to the assay either directly in the form of C99 or C83 or, alternatively, in the form of a full-length βAPP being processed “in situ” to C99 or C83.

Furthermore, “βAPP” may also stand for a fragment and for a modified (e.g. genetically engineered or chemically modified) derivative of full-length βAPP, C99 or C83, as long as such a fragment or derivative may serve as substrate for γ-secretase activity in an assay as described hereafter. Genetically engineered βAPP may be a βAPP in the above defined sense and modified by amino acid substitutions, deletions or insertions, under the proviso that the screening method according to the invention can still be performed therewith. For instance, a modified βAPP not comprising a cleavage site corresponding to the cleavage site identified by the inventors or a modified βAPP targeted to cellular compartments where no γ-secretase activity is present would not be suited to the method of the invention. Furthermore, genetically engineered βAPP might be a βAPP fused with a reporter protein such as green fluorescent protein, luciferase, β-galactosidase and so on.

swAPP₆₉₅ is especially preferred in an assay as described hereafter, because it is a better substrate for β-secretase than wild type APP, thus providing high levels of C99, the immediate substrate of γ-secretase activity.

The term “γ-secretase activity” means in the context of this invention any proteolytic activity that is capable of cleaving the substrate C99 between amino acids 49 and 50 and whose physiological substrate is C99 in its physiological environment (e.g. in cell membranes). Thus, “γ-secretase activity” may be the proteolytic activity presently ascribed to the not yet fully characterized γ-secretase per se, a proteolytic activity of γ-secretase in combination with one or more presenilins or, if applicable, presenilin activity per se.

The term “source of γ-secretase activity” comprises any biological material such as cells, homogenized cells, enriched membrane fractions, purified membranes, protein fractions, proteins reconstituted in membranes etc. which possess γ-secretase activity. Preferably, the source are human embryonic kidney (HEK) 293 cells, more preferably membrane preparations thereof that can be obtained e.g. as described in Example 1. The methods for the purification of membranes are known in the art (cf. e.g. Methods in Enzymology, Vol. 219: “Reconstitution of intracellular Transport” and T. G. Cooper: “Biochemische Arbeitsmethoden”, De Gruyter Verlag, 1981).

The term “substrate of γ-secretase activity” is meant to encompass “βAPP” according to the definition above as well as artificially designed proteins that encompass the cleavage site detected by the inventors. The cleavage site may be defined by the sequence ITLVML (SEQ ID NO: 3) or VIVITLVMLKKK (SEQ ID NO: 4). Preferably, swAPP₆₉₅ (over)expressed in e.g. HEK 293 cells (and endogenously cleaved to C99) is the substrate of γ-secretase activity.

The term “substance capable of modulating γ-secretase activity” means naturally occurring and synthetic compounds capable of activating or inhibiting enzymatic γ-secretase activity as defined above, whereby substances unspecifically interfering with enzymatic reactions (such as e.g. agents that cause denaturation of proteins) should, of course, not be encompassed. Persons skilled in the art will be able to differentiate between specific and unspecific inhibition or activation of enzymatic activity.

The term “fragment beginning with a sequence corresponding to the N-terminal sequence of CTFγ/50” encompasses, of course, CTFγ/50 itself. However, it is understood that if a βAPP modified C-terminal of the cleavage site identified by the inventors is used as a substrate for γ-secretase activity, a modified C-terminal fragment will result that will have to be detected according to the method of the invention. In other words, said fragment originates from proteolytic cleavage of a βAPP as defined above at the cleavage site identified by the inventors, i.e. the site corresponding to amino acids 49 and 50 of C99.

The term “conditions allowing enzymatic activity of γ-secretase activity” means that reaction conditions are chosen under which proteolytic cleavage by γ-secretase activity is enabled. Examples for such conditions are given below.

According to one embodiment of the invention, the substrate of γ-secretase activity is an endogenous βAPP that is constitutively expressed in the cell. According to another embodiment of the invention, cells expressing an exogenous βAPP are used. Especially preferred is exogenous swAPP₆₉₅. Expression of exogenous βAPP may be achieved by transfecting cells with a gene coding for βAPP in a suitable expression vector. Endogenous and exogenous substrates are described in detail in WO 01/16355.

According to a further embodiment of the invention, βAPP substrate is a fusion protein of a reporter protein with e.g. wild-type βAPP, swAPP or C99. Such substrates and their production are described in detail in WO 01/16355. Commonly used reporter proteins are green fluorescent protein, luciferase, β-galactosidase etc.

The cell line used in the example below is HEK 293. However, other cells and cell lines, such as H4, U373, NT2, PC12, COS, CHO, fibroblasts, myeloma cells, neuroblastoma cells, hybridoma cells, oocytes, empryonic stem cells and so on can be used as well. Cells and cell lines of neuronal or glial origin or fibroblasts are especially preferred. Furthermore, cells and tissues of the brain as well as homogenates and membrane preparations thereof may be used.

The detection of CTFγ/50 or of a fragment beginning with a sequence corresponding to the N-terminal sequence of CTFγ/50 can be performed by immunoblotting/western blotting, by ELISA and other suitable peptide or protein detection methods known in the art.

An even higher sensitivity of detection can be achieved by immunoprecipitation with subsequent immunoblotting/western blotting. Respective protocols can be found e.g. in Ida et al. (1996). Antibodies useful for the detection of CTFγ/50 are, e.g., polyclonal antibody 6687 binding to the last 20 C-terminal amino acids of βAPP (Steiner et al., 2000) and SAD3128 available from LABGEN®.

If a βAPP fused to a reporter protein as mentioned above is used, it is possible to estimate the amount of cleaved substrate by detection of the reporter protein. In this case, a separation of cleaved and uncleaved substrate followed by the respective detection method depending on the reporter protein might be performed.

In the method according to the invention, a source of γ-secretase activity and a substrate thereof are incubated with a test substance under conditions allowing enzymatic activity of the source of γ-secretase activity. After the incubation, the amount of substrate cleaved in presence of the test substance is determined by measuring the amount of CTFγ/50 (or a derivative thereof, respectively, if a derivative of C99 or βAPP has been used as the substrate) produced. The amount of CTFγ/50 (or derivative) released during the incubation step reflects the activity of γ-secretase activity acting at the cleavage site of βAPP as defined above. In most cases, control experiments will be included in the assay, wherein the experiment described before will be performed under essentially the same conditions as above but without addition of said test substance or with addition of a substance known to have no effect on γ-secretase activity. In this case, the results of both experiments will be compared and a reduced amount of released CTFγ/50 (or the derivative) will be indicative of an inhibitory effect of the test substance on γ-secretase activity (inhibitor), whereas an increased amount of CTFγ/50 indicates that the test substance activates said activity (activator). In both cases, said test substance is classified as “modulator”.

Concerning the reaction conditions to be applied, a broad range of buffers can be used. Especially, a reaction buffer having a pH in the range of 5 to 10, more preferably in the range of 6 to 8 and most preferably in the range of 6.3 to 6.9 may be used. One example for a suitable buffer is 150 mM sodium citrate, pH 6.4. The reaction temperature may be selected, for example, in the range of 20° C. to 37° C. A higher temperature might result in denaturation of proteins, a lower temperature will result in a decrease of the speed of the reaction. The reaction mixture may additionally comprise membrane stabilizing agents such as sucrose and sorbitol, preferably in an amount of 200 to 1000 mM, more preferably in an amount of 200 to 500 mM and most preferably in an amount of 200 to 300 mM. Moreover, the reaction buffer may contain proteinase inhibitors, e.g. the “PI Complete” mix, available from Roche®, and EDTA. EDTA serves to inhibit metalloproteinases responsible for the degradation of CTFγ. Said proteinase inhibitor mix does not include pepstatin which is an inhibitor of γ-secretase activity.

According to another embodiment of the invention, an assay based on the method according to the invention will be used in a two step analysis of test substances: As mentioned above, several assays are known in the art in which γ-secretase activity is measured by assessing release of Aβ (Wolfe et al., 1999; “Aβ release based assays”). As also mentioned above, Aβ release might be the result of an at least two-step cleavage process: one cleavage occurring at the site identified by the inventors (amino acid 49/50 of C99), the other at site 40/41 (resulting in Aβ940) or site 42/43 (resulting in Aβ142) of C99. Thus, Aβ release based assays do not discriminate between inhibitors (or, more generally, modulators) of these two cleavage activities. By coupling the known Aβ release based assay to the assay according to the invention, it is possible to make said discrimination. For example, it is possible to first identify inhibitors by performing the Aβ release based assay and, afterwards, including test substances having shown to be effective in said first assay in the second assay based on the method according to the invention. Substances not being effective in said second assay can be classified as substances modulating the proteolytic activity acting at amino acids 40/41 or 42/43, whereas substances found to be effective in both assays act on cleavage site 49/50.

The two-step assay outlined above is of great importance because of the following reason: as discovered by the inventors, the cleavage site 49/50 in C99 shows similarity to the known S3 cleavage site of Notch protein (Schroeter et al., 1998). If both cleavage events are mediated by the same or closely related proteinases, it is expected that substances inhibiting cleavage of C99 at site 49/50 might also inhibit Notch protein cleavage, possibly resulting in severe and undesirable side-effects of medicaments ultimately derived from a respective substance. Thus, the novel screening method has the additional advantage that it provides a means for the discrimination between substances that are suspected to affect Notch protein proteolysis and substances not expected to do so.

Yet another important embodiment of the present invention is a method according to the invention, characterized in that said method is a high throughput screening (HTS) method. HTS relates to an experimental setup wherein a large number of compounds is tested simultaneously. Preferably, said HTS setup may be carried out in microplates, may be partially or fully automated and may be linked to electronic devices such as computers for data storage, analysis, and interpretation using bioinformatics. Preferably, said automation may involve robots capable of handling large numbers of microplates and capable of carrying out several thousand tests per day. Preferably, a test compound which is known to show the desired modulating or inhibitory function will also be included in the assay as a positive control. The term HTS also comprises ultra high throughput screening formats (UHTS). Preferably, said UHTS formats may be carried out using 384- or 1536-well microplates, sub-microliter or sub-nanoliter pipettors, improved plate readers and procedures to deal with evaporation. HTS methods are described e.g. in U.S. Pat. No. 5,876,946 and U.S. Pat. No. 5,902,732. The expert in the field can adapt the method described below to a HTS or UHTS format without the need of carrying out an inventive step.

The source of γ-secretase activity (e.g. membrane preparations), the substrate of γ-secretase activity (e.g. C99 or a modified derivative as outlined above), a reaction buffer and a means for specifically detecting the reaction product CTFγ/50 (or a respective derivative) may be assembled to a test kit. The detection means may be a monoclonal or a polyclonal antibody or derivative specifically reacting with CTFγ/50. The test kit may additionally comprise microtiter plates, labeled second antibodies used for immunoblotting/western blotting techniques (known in the art), reaction vessels, blotting membranes, buffers etc.

Although the method according to the invention is useful for the detection of both, inhibitors and activators of γ-secretase activity, screening methods and test kits directed to the identification of inhibitors are of special interest.

Also encompassed by the present invention are substances modulating-secretase activity that will be identified by the method according to the invention and, especially, inhibitors of secretase activity. It is understood that already known inhibitors such at DAPT (N-[N-(3,5-difluorophenacetyl)-L-alanyl]-S-phenylglycin t-butyl ester) and “Compound 1” (N-[(1,1-dimethylethoxy)carbonyl]-L-valyl-(4S,5S)-4-amino-2,2-difluoro-5-methyl-3-oxoheptanoyl-L-valyl-L-Isoleucine methyl ester; also called CM256) will be excluded from the scope of protection.

According to a further aspect of the invention, there are provided pharmaceutical compositions comprising substances identified with the method according to the invention and pharmaceutical acceptable carriers or excipients. A pharmaceutically acceptable carrier can contain physiologically acceptable compounds that act, for example, to stabilize or to increase the absorption of a substance capable of inhibiting γ-secretase activity. Such physiologically acceptable compounds include, for example, carbohydrates such as glucose, sucrose or dextrans, antioxidants such as ascorbic acid or glutathione, chelating agents, low molecular weight proteins or other stabilizers or excipients (as disclosed e.g. in Remington's Pharmaceutical Sciences (1990), 18^{th} ed., Mack Publ., Easton). The person skilled in the art will know that the choice of a pharmaceutically acceptable carrier, including a physiologically acceptable compound, depends, for example, on the route of administration of the composition.

According to yet another aspect of the invention, a substance identified with the method according to the invention is used for the preparation of a medicament for the treatment of neurodegenerative diseases, especially Alzheimer's disease.

Moreover, the invention provides the previously unknown CTFγ/50 (SEQ ID NO: 1). It is clear that allelic variations and functionally equivalent derivatives thereof will also be encompassed by the invention.

### EXAMPLES

### Example 1

### Identification of CTFγ/50

In the Experiments Outlined Below, the Following Materials and Methods Have Been Used:

Cell Lines, Cell Culture and cDNA Transfection

Human embryonic kidney 293 (HEK 293) cells expressing swAPP were generated and cultured as described (Steiner et al., 2000).

For control experiments demonstrating that CTFγ/50 is not identical with CTFγ57, HEK 293 cells were transiently transfected with cDNA encoding recombinant CTFγ57 using DOTAP (liposome formulation of the monocationic lipid N-[1-(2,3-Dioleoyloxy)]-N,N,N-trimethylammonium propane methylsulfate in water; Roche) according to the supplier's instructions.

cDNA Constructs

A cDNA encoding recombinant CTFγ57 was amplified by PCR and cloned into pcDNA3 vector (Invitrogen) as an HindIII and Xbal fragment. Recombinant CTFγ57 consists of an N-terminal methionine followed by the C-terminus of βAPP starting at position 43 of the β-amyloid domain.

Antibodies

The polyclonal antibodies 6687 to the last 20 C-terminal amino acids of βAPP (Steiner et al., 2000) and 3926 to Aβ1-42 (Wild-Bode et al., 1997) have been described. The monoclonal antibody 6E10 to Aβ1-17 was obtained from Senetek, USA.

Inhibition of γ-Secretase Activity

γ-Secretase activity was inhibited using DAPT (N-[N-(3,5-difluorophenacetyl)-L-alanyl]-S-phenylglycin t-butyl ester) (Dovey et al., 2001) and CM256 (N-[(1,1-dimethylethoxy)carbonyl]-L-valyl-(4S,5S)-4-amino-2,2-difluoro-5-methyl-3-oxoheptanoyl-L-valyl-L-Isoleucine methyl ester; gift from Dr. M. Wolfe), previously designated “Compound 1” (Esler et al., 2000), that were diluted from stock solutions in DMSO to the concentrations described.

Analysis of Secreted Aβ

Aβ was immunoprecipitated from conditioned media collected for 4 h with antibody 3926, separated on Tris-Tricine gels and detected by immunoblotting with antibody 6E10 using a chemiluminescent detection system (Tropix, USA).

Analysis of CTFγ in Vivo

CTFγ was analyzed by combined immunoprecipitation/westernblotting with antibody 6687 of membrane extracts from stably transfected HEK 293 cells or from mouse brain. Briefly, homogenates of cells or brain tissue were prepared in hypotonic buffer (10 mM Tris, pH 7.4, 1 mM EDTA, 1 mM EGTA, pH 7.0) containing 1×protease inhibitors (PI) (Complete, Roche) as described (Steiner et al., 1998). Following homogenization membranes were isolated from the postnuclear supernatant (PNS) by centrifugation at 16.000 g for 45 min at 4° C. The membranes were resuspended in RIPA buffer (150 mM NaCl, 10 mM Tris, 1% NP-40, 0.5% cholic acid, 0.1% SDS, 5 mM EDTA, pH 8.0, 1×PI) following a clarifying spin at 16.000 g for 10 min at 4° C., and subjected to immunoprecipitation with antibody 6687.

To analyze expression of recombinant CTFγ57, lysates were prepared with RIPA buffer 48 h after transfection and subjected to immunoprecipitation with antibody 6687.

Generation and Analysis of CTFγ in Vitro

CTFγ was generated in vitro from membrane preparations of HEK 293 cells stably transfected with swAPP following previously described procedures (McLendon et al., 2000; Pinnix et al., 2001). In brief, cells were resuspended (0.5 ml/10-cm dish) in homogenization buffer (10 mM MOPS, pH 7.0, 10 mM KCl, 1×PI). Cell homogenates and a PNS were prepared as described (Steiner et al., 1998). Membranes were pelleted from the PNS by centrifugation for 20 min at 16.000 g at 4° C., washed with homogenization buffer and resuspended (50 μl/0-cm dish) in assay buffer (150 mM sodium citrate, pH 6.4, 1×PI). To allow generation of CTFγ, samples were incubated at 37° C. for the indicated time points in a volume of 25 μl/assay. Control samples were kept on ice. After termination of the assay reactions on ice, samples were separated into pellet (P100) and supernatant (S100) fractions by ultracentrifugation for 1 h at 100.000 g at 4° C. Following SDS-PAGE on 10-20% Tris-Tricine gels (Invitrogen) samples were analyzed for CTFγ by immunoblotting with antibody 6687.

Radiosequencing of CTFγ

Confluent swAPP transfected HEK 293 cells in 10-cm dishes were radioactively labeled with 1.4 mCi/dish ³⁵S-methionine (Promix, Amersham Pharmacia Biotech) for 4 h in methionine-free MEM. CTFγ was then generated in vitro from membrane preparations as described above except that after termination of CTFγ generation the assay reactions were separated into pellet (P16) and supernatant (S16) fractions by centrifugation at 16.000 g for 30 min at 4° C . After isolation of CTFγ from S16 by immunoprecipitation with antibody 6687 immunocomplexes were separated by SDS-PAGE on 10-20% Tris-Tricine gels (Invitrogen) and blotted onto a PVDF membrane. After autoradiography, the CTFγ band was excised and subjected to radiosequencing by automated Edman degradation as described (Haass et al, 1992).

Results:

A mentioned above, PS mediated γ-secretase cleavage does not only result in the generation of soluble Aβ but also in the generation of a βAPP C-terminal fragment (CTFγ) representing the counterpart of Aβ. Based on the known heterogenous C-terminal sequences of Aβ, two species of CTFγ have been postulated, CTFγ/57 and CTFγ/59, respectively.

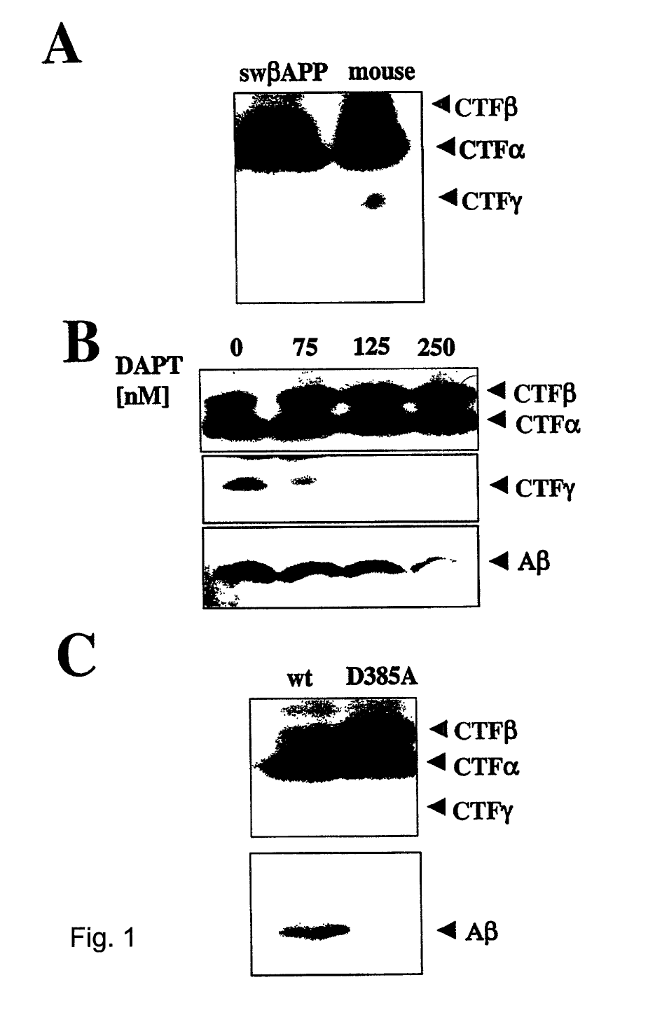

To investigate this cleavage in living cells, C-terminal fragments of βAPP were immunoprecipitated from membrane fractions of human embryonic kidney 293 (HEK 293) cells stably transfected with βAPP₆₉₅ carrying the Swedish mutation (swAPP) (Citron et al., 1992). This revealed the presence of an approximately 6 kDa C-terminal fragment migrating below the major βAPP CTFs generated by α-and β-secretase (FIG. 1A; left panel). A CTF of similar molecular weight was also found in homogenates of mouse brain (FIG. 1A; right panel).

In order to investigate whether this polypeptide represents the γ-secretase generated CTFγ, swAPP transfected cells were treated with the previously described γ-secretase inhibitor DAPT (Dovey et al., 2001). As shown in FIG. 1B, concomitant with an increase of βAPP CTFβ and CTFα (upper panel; CTFα is a cleavage product of the proteolytic activity of alpha-secretase), a dose dependent inhibition of CTFγ generation was observed (middle panel). This was further confirmed by the immunoprecipitation of Aβ from the conditioned media of these cells, which consistent with previous results (Dovey et al., 2001) revealed a severely reduced Aβ production (FIG. 1B; lower panel).

To demonstrate the PS dependency of this cleavage, βAPP and its proteolytic fragments were immunoprecipitated from cells expressing PS1 D385N. As shown previously (Steiner et al., 1999; Wolfe et al., 1999), PS1 D385N acts like a dominant negative mutation that inhibits the biological function of PSs required for the γ-secretase cleavage of βAPP. As expected, a strong increase of βAPP CTFβ and CTFα was observed, indicating a significant inhibition of γ-secretase cleavage (FIG. 1C). Moreover, generation of CTFγ was strongly reduced (FIG. 1C). These results demonstrate that the observed low molecular weight C-terminal cleavage product of βAPP fulfills the criteria of being produced by γ-secretase cleavage. However, rather small amounts of this fragment accumulate in vivo, most likely due to the very rapid degradation of this fragment. Therefore, the inventors attempted to generate CTFγ in an in vitro assay, which could allow the efficient stabilization of this fragment by the use of a variety of protease inhibitors.

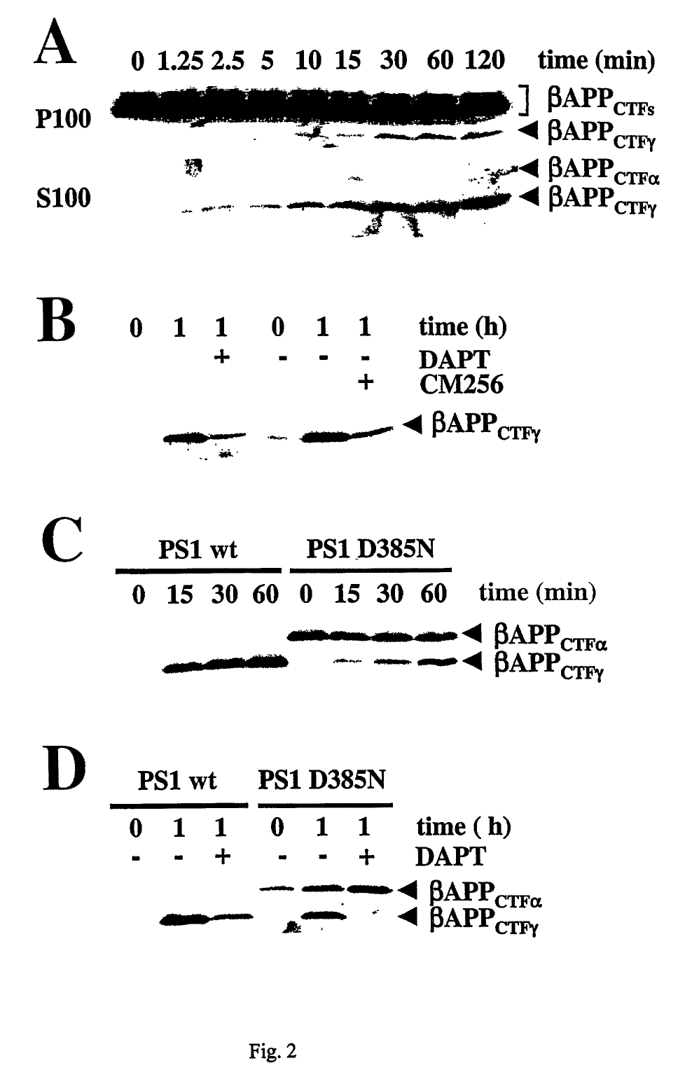

Membranes from HEK 293 cells stably expressing swAPP were incubated under the conditions described under item “Generation and analysis of CTFγ in vitro” above in the presence of a protease inhibitor cocktail (McLendon et al., 2000; Pinnix et al., 2001). After termination of the in vitro assay, membranes were separated by ultracentrifugation. The pellet (P100) and the supernatant (S100) fraction were analyzed for the presence of βAPP CTFs. CTFα and CTFβ were predominantly observed within the P100 fraction (FIG. 2A; upper panel), whereas CTFγ was significantly enriched in the S100 fraction (FIG. 2A; lower panel). The predominant accumulation of CTFγ within the cytoplasmic fraction demonstrates that this fragment is released from the membrane. Prolonged incubation resulted in the generation of robust levels of CTFγ (FIG. 2A, lower panel). The maximum production of CTFγ was observed after approximately 1 to 2 h (FIG. 2A). To show that the in vitro generated CTFγ is indeed the product of an authentic PS dependent γ-secretase cut, the membrane fractions were incubated in the presence of two previously described γ-secretase inhibitors, DAPT (Dovey et al., 2001) and CM256 (Esler et al., 2000). As shown in FIG. 2B, both γ-secretase inhibitors efficiently reduced in vitro generation of CTFγ. Moreover, CTFγ generation was also significantly reduced when membranes were isolated from HEK 293 cells coexpressing swAPP and the functionally inactive PS1 D385N mutation described above (FIG. 2C). The remaining minor production of CTFγ was further reduced by the addition of the γ-secretase inhibitor DAPT (FIG. 2D). Taken together, these results demonstrate that the in vitro assay produces very robust levels of CTFγ in a PS and γ-secretase dependent manner. Moreover, the same fragment is also produced in vivo and can be found in mouse brain.

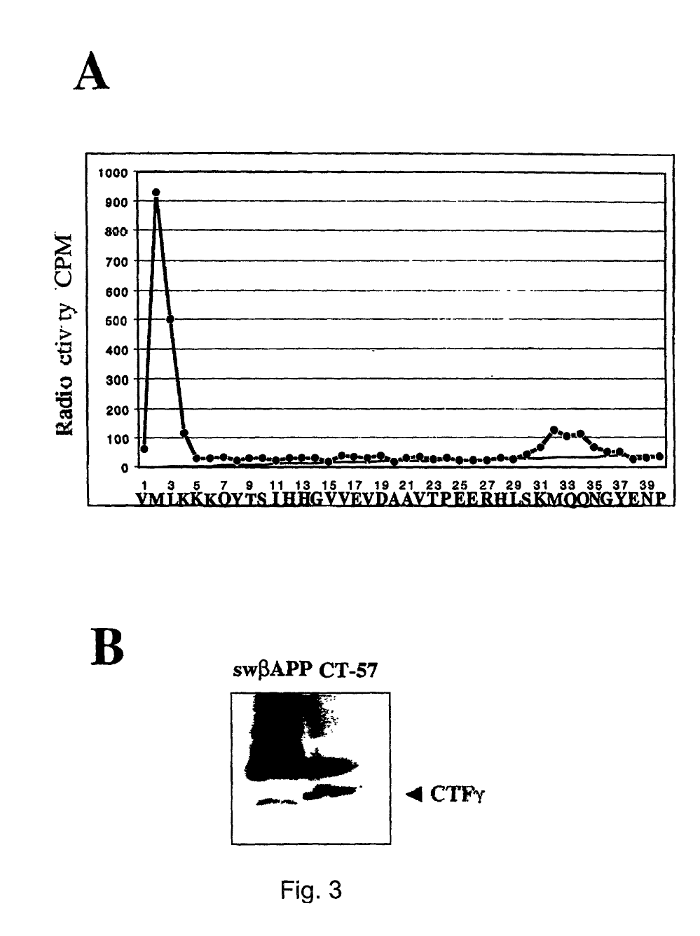

The inventors then used the in vitro assay to isolate sufficient amounts of CTFγ to allow the further structural characterization of this peptide. However, attempts to perform mass spectroscopy with the isolated peptide were unsuccessful for unknown reasons. In a further approach, the inventors tried to determine the sequence of the peptide's N-terminus by radiosequencing. HEK 293 cells stably expressing swAPP were metabolically labeled with ³⁵S-methionine. Radiolabeled CTFγ was generated in vitro as described above. After ultracentrifugation, CTFγ was immunoprecipitated from the S100 fraction with antibody 6687 to the last 20 amino acids of βAPP. Radiolabeled CTFγ was then subjected to automated Edman degradation (Haass et al., 1992). Surprisingly, this revealed a major peak of radioactivity in fraction 2 and not in fractions 9 and 11 as one would have expected for a CTFγ beginning at position 41 or 43 (FIG. 3A). This indicates that CTFγ is generated by a proteolytic cleavage between amino acids 49 and 50 (FIG. 3A). The peak of radioactivity in fraction 2 thus corresponds to methionine 51. Consistent with a proteolytic fragment starting at valine 50, a second peak of radioactivity was observed at position 32 thus confirming that CTFγ predominantly begins with amino acid 50. In order to demonstrate that such a truncated CTFγ is also generated under in vivo conditions (where it is impossible to obtain sufficient amounts for sequencing), we co-migrated a recombinant CTFγ beginning at amino acid 43 with CTFγ produced in living HEK 293 cells. As shown in FIG. 3B, this revealed that in vivo generated CTFγ migrated at a lower molecular weight than the recombinant CTFγ. Together with the experiments described in FIG. 1, this confirms that a major PS dependent cut of βAPP occurs C-terminal of the authentic γ-secretase cleavage after amino acids 40 and 42. Moreover, this also demonstrates that the smaller CTFγ is not simply generated by unspecific degradation, since the recombinant fragment should have been cleaved as well under these conditions.

Thus, biochemical characterization of CTFγ surprisingly revealed a major cut of βAPP₆₉₅ after amino acid 645 (FIG. 3), corresponding to amino acid 49 of the β-amyloid domain of βAPP. This cleavage is fully dependent on biologically active presenilins (FIG. 2). Moreover, two independent γ-secretase inhibitors that were both described to efficiently block Aβ40 and Aβ42 generation also inhibited the cleavage after amino acid 49 of the β-amyloid domain (FIGS. 2B and 2D). Thus it appears likely that this cleavage occurs by the γ-secretase itself. The above results therefore suggest that γ-secretase mediates at least three different cuts within the C-terminal domain of βAPP.

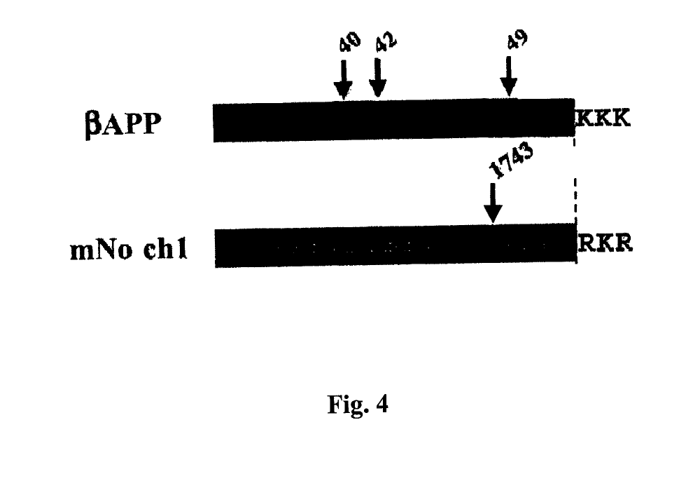

Interestingly, the N-terminus of CTFγ is located at a position, which is homologous to the PS dependent S3 cleavage of Notch (FIG. 4). The S3 cleavage of Notch occurs right at the cytoplasmic boarder of the membrane (Schroeter et al., 1998). Moreover, the novel cleavage site of βAPP may also occur close to the cytoplasmic boarder of the membrane. Based on the results by Tischler et al. (Tischler and Cordell, 1996), the cut between amino acid 49 and 50 would indeed occur within the cytoplasmatic domain. Such a cytoplasmatic cleavage would be more likely than an intramembraneous proteolytic cut, which is likely to be inhibited by the higly hydrophobic environment within a phospholipid bilayer. In fact a cytoplasmic cleavage may facilitate a shift of the remaining stub into the cytoplasm, where it could easily be attacked by the final γ-secretase cut. Indeed, Murphy et al., 1999, provided evidence for such a model. Moreover, this may indicate that the cytoplasmic tail of βAPP requires “shedding” before/during it undergoes the final γ-secretase cut, a phenomenon which would be very similar to the required ectodomain shedding of γ-secretase substrates (Struhl and Adachi, 2000). Furthermore, the identification of CTFγ in vivo may also raise the interesting possibility that this fragment similar the Notch intracellular cytoplasmic domain (NICD) may have a biological function in signal transduction. Based on the striking similarity of the biological mechanisms involved in the generation of NICD and CTFγ as well as potentially similar functions in signal transduction, the term AICD for the Amyloid precursor protein intracellular domain is proposed.

Both cleavages are PS dependent and can be blocked by γ-secretase inhibitors (De Strooper et al., 1999; De Strooper et al., 1998). Thus, it is likely that γ-secretase cleavage at position 49 of βAPP and S3 cleavage of Notch are mediated by the same PS dependent enyzme. The fact that no N-terminal heterogeneity has been observed for both NICD (Schroeter et al., 1998) and CTFγ (data shown here) outlines a further similarity between these two γ-secretase cleavages. Furthermore, PS dependent cleavage of βAPP and Notch at similar sites provides additional evidence for a direct function of presenilins in γ-secretase/S3 cleavage.

The finding of an additional γ-secretase cut close to the predicted border of the transmembrane domain may indicate that the cytoplasmic tail of βAPP requires “shedding” before/during it undergoes the final γ-secretase cut after positions 40/42 of the β-amyloid domain. Alternatively, γ-secretase may cut first at position 40/42 of the β-amyloid domain followed by a second cleavage after position 49 releasing CTFγ from the membrane. However, since neither an Aβ49 species nor a CTFγ starting at position 41/43 of the β-amyloid domain has been found, the data may indicate simultaneous cleavage at all three sites.

### Example 2

### Screening Assay—Medium Throughput

In the following, a screening assay will be described in detail. However, it is to be understood that this invention is not limited to specific assay formats, materials or reagents, as such may, of course, vary. It is also to be understood that the terminology used herein is for the purpose of describing particular embodiments only and is not intended to be limiting.

HEK 293 cells stably transfected with βAPP₆₉₅ carrying the Swedish mutation (swAPP) were grown as described in Citron et al., 1992, i.e. in DMEM with Glutamax (Gibco BRL) containing 10% FCS, 1% Penicillin/Streptavidin, 200 μg/ml G418. The cells were resuspended (0.5 ml/10-cm dish) in homogenization buffer (10 mM MOPS, pH 7.0, 10 mM KCl, 1×PI). Cell homogenates and a post nuclear supernatant (PNS) were prepared by centrifugation at 2500 g for 15 min. Membranes were pelleted from the PNS by centrifugation for 20 min at 16.000 g at 4° C., washed with homogenization buffer and resuspended (50 μl/10-cm dish) in assay buffer (150 mM sodium citrate, pH 6.4, 1×PI). To allow generation of CTFγ, control samples without added test substance and samples comprising different concentrations of substances to be screened were incubated at 37° C. for e.g. 1 h. The reactions were stopped by cooling them to 4° C.

After termination of the reactions, the samples are separated into pellet (P100) and supernatant (S100) fractions by ultracentrifugation for 1 h at 100.000 g at 4° C. CTFγ/50 is detected and quantified by ELISA using an antibody specific for the N-terminal sequence of CTFγ/50. A reduced signal compared to the control sample indicates that the respective test compound inhibits γ-secretase activity acting at amino acid 49/50 of C99.

### Example 3

### Screening Assay—Medium Throughput

The screening assay is carried out as described in Example 2 with the exception that instead of the ultracentrifugation step membranes are solubilized by adding STEN—lysis buffer (50 mM Tris, pH 7.6; 150 mM NaCl; 2 mM EDTA; 1% NP-40 (final)). CTFγ/50 is detected and quantified in this solution by ELISA as described in Example 2.

### Example 4

### Screening Assay—Low Throughput

The screening assay is carried out as described in Example 2 with the exception that the release of CTFγ/50 is determined by immunoprecipitation and immunoblotting of CTFγ/50 and densitometric analysis of the resulting bands. A reduced signal compared to the control sample indicates that the respective test compound inhibits γ-secretase activity acting at amino acid 49/50 of C99.

References

Citron, M., Oltersdorf, T., Haass, C., McConlogue, L., Hung, A. Y., Seubert, P., Vigo-Pelfrey, C., Lieberburg, I. and Selkoe, D. J. (1992) Mutation of the β-amyloid precursor protein in familial Alzheimer's disease increases β-protein production. Nature, 360, 672-674.

De Strooper, B., Annaert, W., Cupers, P., Saftig, P., Craessaerts, K., Mumm, J. S., Schroeter, E. H., Schrijvers, V., Wolfe, M. S., Ray, W. J., Goate, A. and Kopan, R. (1999) A presenilin-1-dependent γ-secretase-like protease mediates release of Notch intracellular domain. Nature, 398, 518-522.

De Strooper, B., Saftig, P., Craessaerts, K., Vanderstichele, H., Guhde, G., Annaert, W., Von Figura, K. and Van Leuven, F. (1998) Deficiency of presenilin-1 inhibits the normal cleavage of amyloid precursor protein. Nature, 391, 387-390.

Dovey, H. F., John, V., Anderson, J. P., Chen, L. Z., de Saint Andrieu, P., Fang, L. Y., Freedman, S. B., Folmer, B., Goldbach, E., Holsztynska, E. J., Hu, K. L., Johnson-Wood, K. L., Kennedy, S. L., Kholodenko, D., Knops, J. E., Latimer, L. H., Lee, M., Liao, Z., Lieberburg, I. M., Motter, R. N., Mutter, L. C., Nietz, J., Quinn, K. P., Sacchi, K. L., Seubert, P. A., Shopp, G. M., Thorsett, E. D., Tung, J. S., Wu, J., Yang, S., Yin, C. T., Schenk, D. B., May, P. C., Altstiel, L. D., Bender, M. H., Boggs, L. N., Britton, T. C., Clemens, J. C., Czilli, D. L., Dieckman-McGinty, D. K., Droste, J. J., Fuson, K. S., Gitter, B. D., Hyslop, P. A., Johnstone, E. M., Li, W. Y., Little, S. P., Mabry, T. E., Miller, F. D. and Audia, J. E. (2001) Functional γ-secretase inhibitors reduce b-amyloid peptide levels in brain. J Neurochem, 76, 173-181.

Esler, W. P., Kimberly, W. T., Ostaszewski, B. L., Diehl, T. S., Moore, C. L., Tsai, J.-Y.,Rahmati T., Xia, W., Selkoe, D. J. and Wolfe, M. S. (2000) Transition-state analogue inhibitors of γ-secretase bind directly to presenilin-1. Nature Cell Biol, 2, 428-433.

Haass, C., Schlossmacher, M. G., Hung, A. Y., Vigo-Pelfrey, C., Mellon, A., Ostaszewski, B. L., Lieberburg, I., Koo, E. H., Schenk, D., Teplow, D. B. and Selkoe, D. J. (1992) Amyloid β-peptide is produced by cultured cells during normal metabolism. Nature, 359, 322-325.

Haass, C. and Selkoe, D. J. (1993) Cellular processing of β-amyloid precursor protein and the genesis of amyloid β-peptide. Cell, 75, 1039-1042.

Ida et al. (1996). J. Biol. Chem. 271, 22908-22914.

McLendon, C., Xin, T., Ziani-Cherif, C., Murphy, M. P., Findlay, K. A., Lewis, P. A., Pinnix, I., Sambamurti, K., Wang, R., Fauq, A. and Golde, T. E. (2000) Cell-free assays for γ-secretase activity. Faseb J, 14, 2383-2386.

Murphy M P, Hickman L J, Eckman C B, Uljon S N, Wang R, Golde T E (1999) gamma-Secretase, evidence for multiple proteolytic activities and influence of membrane positioning of substrate on generation of amyloid beta peptides of varying length. J. Biol. Chem. Apr. 23, 1999 ;274(17):11914-23.

Pinnix, I., Musunuru, U., Tun, H., Sridharan, A., Golde, T., Eckman, C., Ziani-Cherif, C., Onstead, L. and Sambamurti, K. (2001) A novel γ-secretase assay based on detection of the putative C-terminal fragment-γ of amyloid β protein precursor. J Biol Chem, 276, 481-487.

Schroeter, E. H., Kisslinger, J. A. and Kopan, R. (1998) Notch-1 signalling requires ligand-induced proteolytic release of intracellular domain. Nature, 393, 382-386.

Selkoe, D. J. (1999) Translating cell biology into therapeutic advances in Alzheimer's disease. Nature, 399, A23-31.

Steiner, H., Capell, A., Pesold, B., Citron, M., Kloetzel, P. M., Selkoe, D. J., Romig, H., Mendla, K. and Haass, C. (1998) Expression of Alzheimer's disease-associated presenilin-1 is controlled by proteolytic degradation and complex formation. J Biol Chem, 273, 32322-32331.

Steiner, H. and Haass, C. (2000) Intramembrane proteolysis by presenilins. Nature Reviews Molecular Cell Biology, 1, 217-224.

Steiner, H., Kostka, M., Romig, H., Basset, G., Pesold, B., Hardy, J., Capell, A., Meyn, L., Grim, M. G., Baumeister, R., Fechteler, K. and Haass, C. (2000) Glycine 384 is required for presenilin-1 function and is conserved in polytopic bacterial aspartyl proteases. Nature Cell Biol, 2, 848-851.

Steiner, H., Romig, H., Pesold, B., Philipp, U., Baader, M., Citron, M., Loetscher, H., Jacobsen, H. and Haass, C. (1999) Amyloidogenic function of the Alzheimer's disease-associated presenilin 1 in the absence of endoproteolysis. Biochemistry, 38,14600-14605.

Struhl, G. and Adachi, A. (2000) Requirements for presenilin-dependent cleavage of Notch and other transmembrane proteins. Mol Cell, 6, 625-636.

Tischer E, Cordell B. (1996) Beta-amyloid precursor protein. Location of transmembrane domain and specificity of gamma-secretase cleavage. J Biol Chem. Sep.6, 1996 6;271(36):21914-9.

Vassar, R. and Citron, M. (2000) Ab-generating enzymes: recent advances in β- and γ-secretase research. Neuron, 27, 419-422.

Wild-Bode, C., Yamazaki, T., Capell, A., Leimer, U., Steiner, H., Ihara, Y. and Haass, C. (1997) Intracellular generation and accumulation of amyloid β-peptide terminating at amino acid 42. J Biol Chem, 272, 16085-16088.

Wolfe, M. S. and Haass, C. (2001) The role of presenilins in g-secretase activity. J Biol Chem, 276, 5413-5416.

Wolfe, M. S., Xia, W., Ostaszewski, B. L., Diehl, T. S., Kimberly, W. T. and Selkoe, D. J. (1999) Two transmembrane aspartates in presenilin-1 required for presenilin endoproteolysis and γ-secretase activity. Nature, 398, 513-517.

1. A method of screening for substances capable of modulating γ-secretase activity comprising the steps:
(a) providing a source of γ-secretase activity,
(b) providing a substrate for γ-secretase activity, said substrate comprising a cleavage site corresponding to the naturally occurring cleavage site between amino acid 49 and amino acid 50 of the γ-secretase substrate C99 (SEQ ID NO:2),
(c) incubating said source and said substrate with a test substance under conditions allowing enzymatic activity of γ-secretase activity, and
(d) determining γ-secretase activity by detecting a C-terminal fragment released from said substrate, wherein said C-terminal fragment is a fragment beginning with a sequence corresponding to the N-terminal sequence of CTFγ/50 (SEQ. ID NO: 1).

2. The method according to claim 1, comprising the additional step of comparing said determined γ-secretase activity to the γ-secretase activity obtained upon carrying out said incubation in the absence of said test substance.

3. The method according to claim 1, wherein the test substance is screened for an inhibitory effect on γ-secretase activity.

4. The method according to claim 2, wherein the test substance is screened for an inhibitory effect on γ-secretase activity.

5. The method according to claim 1, wherein the source of γ-secretase activity is a membrane of a human embryonic kidney (HEK) 293 cell.

6. The method according to claim 1, wherein said substrate of γ-secretase activity is selected from human wild type βAPP₇₇₀, wild type βAPP₆₉₅, swAPP₆₉₅, C99 and C83.

7. The method according to claim 1, wherein said step of determining γ-secretase activity comprises measuring the release of CTFγ/50.

8. The method according to claim 7, wherein CTFγ/50 is measured by immunoblotting or ELISA.

9. The method according to claim 1, wherein the test substance is a substance showing an inhibitory effect in an Aβ release based assay.

10. The method according to claim 1, wherein the method is carried out in a high throughput screening (HTS) format.

11. A kit for the identification of substances capable of inhibiting γ-secretase activity comprising:
(a) a source of γ-secretase activity,
(b) a substrate for γ-secretase activity, said substrate comprising a cleavage site corresponding to the naturally occurring cleavage site between amino acid 49 and amino acid 50 of the γ-secretase substrate C99 (SEQ ID NO:2),
(c) a reaction buffer, and
(d) a means for specifically detecting a C-terminal fragment released from said substrate, wherein said C-terminal fragment is a fragment beginning with a sequence corresponding to the N-terminal sequence of CTFγ/50 (SEQ ID NO:1).

12. A method of identifying a substance capable of inhibiting γ-secretase activity comprising using the method of claim 1.

13. A method of identifying a substance capable of inhibiting γ-secretase activity comprising using the kit of claim 11.

14. A substance capable of inhibiting γ-secretase activity, identified using a method according to claim 1.

15. A substance capable of inhibiting γ-secretase activity, identified using a kit according to claim 11.

16. A pharmaceutical composition comprising a substance according to claim 14.

17. A pharmaceutical composition comprising a substance according to claim 15.

18. A method of treating a neurodegenerative disease in a patient in need thereof comprising administering to said patient a therapeutically effective amount of a substance according to claim 14.

19. A method of treating a neurodegenerative disease in a patient in need thereof comprising administering to said patient a therapeutically effective amount of a substance according to claim 15.

20. A polypeptide having the amino acid sequence shown in SEQ ID NO:1.
